(19) United States
(12) Patent Application Publication
(10) Pub. No.: US 2013/0112219 A1
(43) Pub. Date: May 9, 2013

(54) ESSENTIALLY AMMONIA-FREE WAVING AGENT

(71) Applicant: Henkel AG & Co. KGaA, Duesseldorf (DE)

(72) Inventors: Birgit Rautenberggroth, Ellerau (DE); Yvonne Lissner, Hamburg (DE)

(73) Assignee: HENKEL AG & CO. KGAA, Duesseldorf (DE)

(21) Appl. No.: 13/706,443

(22) Filed: Dec. 6, 2012

Related U.S. Application Data
(63) Continuation of application No. PCT/EP2011/058878, filed on May 31, 2011.

(30) Foreign Application Priority Data
Jun. 11, 2010 (DE) 102010029976.6

(51) Int. Cl. A61K 8/46; A45D 7/04

(52) U.S. Cl. CPC A61K 8/46; A45D 7/04
USPC 132/204; 424/70.51; 132/314

(57) ABSTRACT

Water-containing agents for the permanent deformation of keratin-containing fibers, in particular human hair, that has a pH from about 8.0 to about 8.5 and contains no more than about 0.1 mmol ammonia per 100 g of the agent, containing (a) about 0.5 to about 20 wt % cysteine, (b) about 0.5 to about 10 wt % of a (C₂ to C₆) alkanolamine as an alkalizing agent, exhibit an outstanding waving effect and are gentle on the skin and less odor-intensive.

### CROSS-REFERENCE TO RELATED APPLICATION

This is a continuation of International Application No. PCT/EP2011/058878, filed May 31, 2011, which was published under PCT Article 21(2) and which claims priority to German Application No. 102010029976.6, filed Jun. 11, 2010, which are all hereby incorporated in their entirety by reference.

### TECHNICAL FIELD

The technical field relates to substantially ammonia-free, preferably ammonia-free, waving agents having a pH greater than 7, containing per 100 g of the agent 30 mmol to 165 mmol of at least one carboxyl-group-containing resp. carboxylate-group-containing thiol compound, as well as at least one (C₂ to C₆) alkanolamine.

### BACKGROUND

The permanent deformation of keratin fibers is usually carried out in such a way that the fibers are mechanically deformed and the deformation is fixed in place by appropriate aids. Before and/or after this deformation, the fibers are treated with the aqueous preparation of a keratin-reducing substance, and after a contact time are rinsed with water or an aqueous solution. In a second step, the fibers are then treated with the aqueous preparation of an oxidizing agent. After a contact time this, too, is rinsed out, and the fibers are released from the mechanical deformation aids (rollers, curlers).

The aqueous preparation of the keratin-reducing substance is usually adjusted to be alkaline, so that on the one hand a sufficient proportion of the thiol functions is present in deprotonated form, and on the other hand the fiber swells and thus allows deep penetration of the keratin-reducing substance into the fiber. The keratin-reducing substance cleaves a portion of the disulfide bonds of the keratin to yield —SH groups, thus resulting in a loosening of the peptide crosslinking and consequently of the tension of the fiber due to the mechanical deformation, yielding a reorientation of the keratin structure. Disulfide bonds are re-linked under the influence of the oxidizing agent, and the keratin structure is thereby re-immobilized in the stipulated deformation. Permanent wave treatment of human hair represents one known method of this kind. This can be applied both to generate curls and waves in straight hair, and to straighten frizzy hair.

The alkaline pH of the waving agent is usually brought about by adding ammonia. Ammonia particularly promotes hair swelling, and thereby additionally assists penetration of the keratin-reducing substance and the quality of the waving action. Unfortunately, especially in persons with sensitive skin, the scalp can be irritated mostly by the ammonia but also by the keratin-reducing substances. Waving agents having ammonia-based alkalizing agents are furthermore accompanied by an odor that is perceived as unpleasant.

It is worthwhile to minimize or, optimally, to entirely eliminate both of the negative accompanying phenomena, without impairing waving effectiveness.

Accordingly, it is desirable to provide an agent for the permanent deformation of keratin-containing fibers in the context of whose use the aforesaid undesired side effects are reduced or entirely precluded. At the same time, a very good waving effect is to be achieved.

### DETAILED DESCRIPTION

It has been found, surprisingly, that in the context of a limited quantity of ammonia in waving agents, a simultaneous use of carboxylate-group-containing thiol compounds as a keratin-reducing substance contributes to achieving the desired outcome. This outcome is attained even with corresponding formulations having no ammonia.

In an exemplary embodiment, a water-containing agent for the permanent deformation of keratin-containing fibers, in particular human hair, has a pH greater than about 7 and no more than about 0.1 mmol ammonia per 100 g of the agent, and contains
(a) per 100 g of the agent, 30 mmol to 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂;
M denotes a hydrogen atom or an equivalent of a mono- or polyvalent cation,
and
(b) at least one (C₂ to C₆) alkanolamine as an alkalizing agent.

The agents contemplated herein have an ammonia content of no more than about 0.1 mmol per about 100 g of the agent according to an embodiment. This means that the agents are substantially ammonia-free, and are to contain only 0 to about 0.1 mmol ammonia per 100 g of the agent.

The agents according to another embodiment preferably have, mutatis mutandis, an ammonia content of no more than about 0.01 mmol, very particularly preferably no more than about 0.001 mmol, very particularly preferably no more than about 0.0001 mmol, based in each case on about 100 g of the agent. Most preferably, the agents are free of ammonia.

In alkaline systems, the addition of compounds containing ammonium ions, i.e. of (NH₄⁺)-containing compounds, can cause ammonia to form. The agents in an embodiment therefore preferably have an (NH₄⁺) ion content of no more than about 0.1 mmol per about 100 g of the agent. This means that the agents are preferably substantially (NH₄⁺) ion-free, and are to contain only 0 to about 0.1 mmol (NH₄⁺) ions per about 100 g of the agent.

The agents according to an embodiment preferably have, mutatis mutandis, an (NH₄⁺) ion content of no more than 0.01 mmol, very particularly preferably no more than 0.001 mmol, very particularly preferably no more than 0.0001 mmol, based in each case on 100 g of the agent. Most preferably, the agents are free of (NH₄⁺) ions.

It has become apparent that the agents contemplated herein achieve the desired effects particularly well when the pH is equal to about 7.5 to about 10.0, in particular about 8.0 to about 9.5.

The agents in a further embodiment contain a carboxylate-group-containing thio compound of formula (I). The aliphatic hydrocarbon skeleton X of formula (I) therein can be saturated or unsaturated, linear or branched.

According to formula (I) it is preferred if X denotes methylene, ethane-1,1-diyl, ethane-1,2-diyl, propane-1,1-diyl, propane-1,2-diyl, propane-1,3-diyl, such that each of these groups optionally can additionally be substituted with at least one of the following residues: thiol group, carboxyl group, carboxylate group, hydroxy group, —NH₂.

In formula (I), X particularly preferably denotes methylene or ethane-1,1-diyl, such that each of these groups optionally can additionally be substituted with at least one of the following residues: carboxyl group, carboxylate group, hydroxy group, —NH₂.

If the compounds of formula (I) are present as a salt, M denotes an equivalent of a mono- or polyvalent cation. The mono- or polyvalent cation M^{z+} having a charge number z of one or higher serves, solely for reasons of electroneutrality, to compensate for the single negative charge of the carboxylate fragment —COO⁻ in formula (I) present in the context of salt formation. The equivalent of the corresponding cation to be used for this is 1/z. In the case of salt formation, the —COOM fragment of formula (I) denotes the group: —COO⁻ 1/z (M^{z+}).

All physiologically acceptable cations are suitable in principle as a mono- or polyvalent cation M. These are, in particular, metal cations of the physiologically acceptable metals from groups Ia, Ib, IIa, IIb, IIIb, VIa, or VIII of the periodic table of the elements, as well as cationic organic compounds having a quaternized nitrogen atom. The latter are formed, for example, by protonation of primary, secondary, or tertiary organic amines with an acid, for example with compounds of formula (I) in their acid form, or by permanent quaternization of the organic amines. (C₂ to C₆) alkanolammonium ions are preferred thereamong. Examples of these cationic organic (C₂ to C₆) alkanolammonium ions are 2-ammonioethanol and 2-trimethylammonioethanol.

M in formula (I) preferably denotes a hydrogen atom, a (C₂ to C₆) alkanolammonium ion, an alkali metal ion, a half-equivalent of an alkaline earth metal ion, or a half-equivalent of a zinc ion, particularly preferably a hydrogen atom or a (C₂ to C₆) alkanolammonium ion.

The term “(C₂ to C₆) alkanolammonium ion” is to be understood herein as organic ammonium compounds that possess two to six carbon atoms, which form a carbon skeleton onto which at least one ammonio group (preferably exactly one ammonio group) and at least one hydroxy group (again, preferably exactly one hydroxy group) are bonded.

Preferably suitable (C₂ to C₆) alkanolammonium ions are 2-ammonioethan-1-ol, 3-ammoniopropan-1-ol, 4-ammoniobutan-1-ol, 5-ammoniopentan-1-ol, 1-ammoniopropan-2-ol, 1-ammoniobutan-2-ol, 1-ammoniopentan-2-ol, 1-ammoniopentan-3-ol, 1-ammoniopentan-4-ol, 3-ammonio-2-methylpropan-1-ol, 1-ammonio-2-methylpropan-2-ol, 3-ammoniopropane-1,2-diol, or 2-ammonio-2-methylpropane-1,3-diol. Very particularly preferably, M in formula (I) denotes a hydrogen atom, 2-ammonioethan-1-ol, or 2-ammonio-2-methylpropan-1-ol.

Preferred agents contain as a compound of formula (I) at least one compound of the group that is constituted from thioglycolic acid, thiolactic acid, cysteine, thiomalic acid, and the salts of the aforesaid thio acids. Preferably suitable compounds of formula (I) are selected from among the salts of thioglycolic acid and/or the salts of thiolactic acid and/or thioglycolic acid and/or thiolactic acid.

The compounds of formula (I) are contained in the waving agents preferably in a quantity from about 40 mmol to about 130 mmol, in particular from about 50 mmol to about 120 mmol, based in each case on 100 g of the agent.

It is moreover preferred if the agents contemplated herein additionally contain no further keratin-reducing compounds besides the compounds of formula (I). Keratin-reducing compounds are capable of reductively cleaving the disulfide bridges of the keratin.

The term “(C₂ to C₆) alkanolamine” is to be understood herein as organic amine compounds that possess a carbon skeleton made up of two to six carbon atoms, onto which at least one amino group (preferably exactly one amino group) and at least one hydroxy group (again, preferably exactly one hydroxy group) are bonded.

The (C₂ to C₆) alkanolamines of the alkalizing agent combination (b) are preferably primary amines.

In the context herein, it is preferred to use at least one (C₂ to C₆) alkanolamine having exactly one amino group. This is, again, preferably a primary amine.

The agent contemplated herein preferably contains at least one (C₂ to C₆) alkanolamine selected from among 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol (in particular from among 2-aminoethan-1-ol, 2-amino-2-methylpropan-1-ol, and 2-amino-2-methyl-propane-1,3-diol).

Monoethanolamine has proven to be a very particularly suitable (C₂ to C₆) alkanolamine as an alkalizing agent.

The agents according to an embodiment contain the (C₂ to C₆) alkanolamines preferably in a quantity of from about 0.5 wt % to about 5.0 wt %, in particular from about 0.7 wt % to about 2.0 wt %, based in each case on the weight of the total agent.

It is further preferred according to an embodiment to additionally use as a further alkalizing agent, besides at least one (C₂ to C₆) alkanolamine, at least one compound that encompasses at least one HCO₃⁻ ion and/or at least one CO₃²⁻ ion. The agent of this embodiment consequently encompasses an alkalizing-agent combination made up of
(b1) at least one (C₂ to C₆) alkanolamine, and
(b2) at least one compound that encompasses at least one HCO₃⁻ ion and/or at least one CO₃²⁻ ion.

The additional alkalizing agent (b2) is preferably selected from among Na₂CO₃, NaHCO₃, K₂CO₃, KHCO₃, CaCO₃, or mixtures thereof (in particular from among Na₂CO₃, NaHCO₃, or mixtures thereof).

Particularly advantageous properties have been found in waving agents in which the alkalizing agents (b1) and (b2) were present at a weight ratio of (b1) to (b2) from about 1 to about 0.1 to about 1 to about 5. Weight ratios from about 1 to about 1.5 to about 1 to about 2.5, in particular of about 1 to about 2, have proven particularly suitable.

The following embodiments (A) to (FF) are preferred embodiments of the agent contemplated herein:

(A):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds according to formula (I)

HS−X−COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one (C₂ to C₆) alkanolamine as an alkalizing agent.

(B):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one (C₂ to C₆) alkanolamine as an alkalizing agent.

(C):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one (C₂ to C₆) alkanolamine as an alkalizing agent.

(D):
An agent for the permanent deformation of keratin-containing fibers, in particular human hair, that has a pH from about 7.5 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one (C₂ to C₆) alkanolamine as an alkalizing agent.

(E):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one (C₂ to C₆) alkanolamine as an alkalizing agent.

(F):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one (C₂ to C₆) alkanolamine as an alkalizing agent.

(G):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one (C₂ to C₆) alkanolamine as an alkalizing agent.

(H):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one (C₂ to C₆) alkanolamine as an alkalizing agent.

(I):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one (C₂ to C₆) alkanolamine as an alkalizing agent.

(J):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one (C₂ to C₆) alkanolamine as an alkalizing agent.

(K):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one (C₂ to C₆) alkanolamine as an alkalizing agent.

(L):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one (C₂ to C₆) alkanolamine as an alkalizing agent.

(M):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one (C₂ to C₆) alkanolamine as an alkalizing agent.

(N):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one (C₂ to C₆) alkanolamine as an alkalizing agent.

(O):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one (C₂ to C₆) alkanolamine as an alkalizing agent.

(P):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄₊) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one (C₂ to C₆) alkanolamine as an alkalizing agent.

(Q):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(R):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(S):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(T):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or an equivalent of a mono- or polyvalent cation,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(U):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(V):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(W):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(X):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(Y): An agent for the permanent deformation of keratin-containing fibers, in particular human hair, that has a pH greater than about 7 and contains no more than about 0.001 mmol ammonia per 100 g of the agent, containing (a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(Z):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(AA):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(BB):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds according to formula (I)

HS—X—COOM (I)

in which
X signifies an aliphatic hydrocarbon skeleton that optionally is additionally substituted with at least one of the following groups: thiol group, carboxyl group, carboxylate group, hydroxy group, —NH₂
M denotes a hydrogen atom or a (C₂ to C₆) alkanolammonium ion,
and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(CC):
An agent for the permanent deformation of keratin-containing fibers, in particular human hair, that has a pH greater than about 7 and contains no more than about 0.001 mmol ammonia per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(DD):
An agent for the permanent deformation of keratin-containing fibers, in particular human hair, that has a pH greater than about 7 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(EE):
An agent for the permanent deformation of keratin-containing fibers, in particular human hair, that has a pH from about 7.5 to about 10.0 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

(FF):
An agent for the permanent deformation of keratin-containing fibers, in particular human hair, that has a pH from about 8.0 to about 9.5 and
(i) contains no more than about 0.1 mmol ammonia per 100 g of the agent, and
(ii) contains no more than about 0.1 mmol (NH₄⁺) ions per 100 g of the agent, containing
(a) per 100 g of the agent, about 30 mmol to about 165 mmol of one or more compounds from the group that is constituted from thioglycolic acid, thiolactic acid, cysteine, thiomalic acid, and the (C₂ to C₆) alkanolammonium salts of the aforesaid thio acids,
   and
(b) at least one alkalizing-agent combination made up of
   (b1) at least one (C₂ to C₆) alkanolamine and
   (b2) at least one compound that encompasses at least one HCO₃⁻ ion and/or at least one CO₃²⁻ ion.

All the aforementioned agents of embodiments (A) to (FF) are preferably free of ammonia and free of (NH₄⁺) ions.

In all the aforesaid agents of embodiments (A) to (FF), it is preferred to select the (C₂ to C₆) alkanolamines used as an alkalizing agent from among 2-aminoethan-1-ol, 2-amino-2-methylpropan-1-ol, and 2-amino-2-methylpropane-1,3-diol. This in turn applies to a particularly preferred extent when these agents (A) to (FF) are free of ammonia and free of (NH₄⁺) ions.

The agents contemplated herein can furthermore contain waving-power-reinforcing components such as, for example:
Heterocyclic compounds such as imidazole, pyrrolidine, piperidine, dioxolan, dioxane, morpholine, and piperazine, as well as derivatives of said compounds such as, for example, the C₁₋₄ alkyl derivatives, C₁₋₄ hydroxyalkyl derivatives, and C₁₋₄ aminoalkyl derivatives. Preferred substituents, which can be positioned both on carbon atoms and on nitrogen atoms of the heterocyclic ring systems, are methyl, ethyl, β-hydroxyethyl, and β-aminoethyl groups. Derivatives preferred of heterocyclic compounds are, for example, 1-methylimidazole, 2-methylimidazole, 4(5)-methylimidazole, 1,2-dimethylimidazole, 2-ethylimidazole, 2-isopropylimidazole, N-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine. Imidazole derivatives that are furthermore preferred herein are biotin, hydantoin, and benzimidazole. Imidazole is very particularly preferred.
Amino acids such as, in particular, arginine, citrulline, histidine, ornithine, and lysine. The amino acids can be used both as a free amino acid and as salts, e.g. as hydrochlorides. Oligopeptides made up of an average of two to three amino acids, and which have a high proportion (>50%, in particular >70%) of the aforesaid amino acids, have further proven to be usable herein. Arginine, as well as salts thereof and arginine-rich oligopeptides, are particularly preferred.
Diols such as, for example, 2-ethyl-1,3-hexanediol, 1,3-butanediol, 1,4-butanediol, 1,2-propanediol, 1,3-propanediol, neopentyl glycol, and ethylene glycol. 1,3-Diols, in particular 2-ethyl-1,3-hexanediol and 1,3-butanediol, have proven to be particularly well suited.

With regard to more detailed information about such waving-power-intensifying components, reference is made to the documents DE Application 44 36 065 and EP-B1-363-057, the content of which is expressly referred to.

The waving-power-intensifying compounds can be contained in the agents contemplated herein in quantities from about 0.5 to about 5 wt %, based on the total agent. Quantities from about 1 to about 4 wt %, in the case of the diols from about 0.5 to about 3 wt %, have proven to be sufficient, and these quantities are therefore particularly preferred.

The agents contemplated herein can additionally contain at least one oil component.

Oil components suitable are, in principle, all oils and fatty substances, as well as mixtures thereof with solid paraffins and waxes. Those oil components whose solubility in water at 20° C. is less than about 1 wt %, in particular less than about 0.1 wt %, are preferred. The melting point of the individual oil or fat components is preferably below approximately 40° C. Oil components that are liquid at room temperature, i.e. below about 25° C., can be particularly preferred. If multiple oil and fat components, as well as optionally solid paraffins and waxes, are used, however, it is generally also sufficient if the mixture of the oil and fat components, as well as optionally paraffins and waxes, meets these requirements.

A preferred group of oil components is vegetable oils. Examples of such oils are apricot-kernel oil, avocado oil, sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.

A further, particularly preferred group of oil components usable herein is liquid paraffin oils and synthetic hydrocarbons, as well as di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, can be preferred.

Fatty acid esters and fatty alcohol esters are likewise oil components usable herein. The monoesters of fatty acids with alcohols having 3 to 24 carbon atoms are preferred. This group of substances refers to the products of esterification of fatty acids having 8 to 24 carbon atoms, such as e.g. hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof that occur, for example, upon high-pressure cleavage of natural fats and oils, upon reduction of aldehydes from Roelen oxosynthesis, or upon dimerization of unsaturated fatty acids, with alcohols such as, for example, isopropyl alcohol, glycerol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol, as well as industrial mixtures thereof that occur, for example, upon high-pressure hydrogenation of industrial methyl esters based on fats and oils or aldehydes from Roelen oxosynthesis, and as a monomer fraction upon dimerization of unsaturated fatty alcohols. Particularly preferred are isopropyl myristate, isononanoic acid C16-18 alkyl esters (Cetiol® SN), stearic acid 2-ethylhexyl ester (Cetiol® 868), cetyl oleate, glycerol tricaprylate, coconut fatty alcohol caprinate/caprylate, and n-butyl stearate.

Dicarboxylic acid esters such as di-n-butyl adipate, di(2-ethylhexyl) adipate, di(2-ethylhexyl) succinate, and diisotridecyl acelaate, as well as diol esters such as ethylene glycol dioleate, ethylene glycol diisotridecanoate, propylene glycol di(2-ethyl hexanoate), propylene glycol diisostearate, propylene glycol dipelargonate, butanediol diisostearate, and neopentyl glycol dicaprylate, also represent oil components usable herein, as do complex esters such as, for example, diacetyl glycerol monostearate.

Oil components preferably usable herein are, lastly, also silicone oils, in particular dialkyl- and alkylarylsiloxanes, for example dimethylpolysiloxane and methylphenylsiloxane, as well as alkoxylated and quaternized analogs thereof as well as cyclic siloxanes. Examples of such silicones are the products marketed by Dow Corning under the designations DC 190, DC 200, and DC 1401, as well as the commercial products DC 344 and DC 345 of Dow Corning, Q2-7224 (manufacturer: Dow Corning; a stabilized trimethylsilylamodimethicone), Dow Corning® 929 Emulsion (containing a hydroxylamino-modified silicone that is also referred to as Amodimethicone), SM-2059 (manufacturer: General Electric), SLM-55067 (manufacturer: Wacker), as well as Abil-Quat 3270 and 3272 (manufacturer: Th. Goldschmidt; diquaternary polydimethylsiloxanes, Quaternium-80). Silicones having a kinematic viscosity of up to about 50,000 cSt measured at 25° can be preferred for purposes herein. Silicone oils having kinematic viscosities of up to 10,000 cSt measured at 25° C. are very particularly preferred. The viscosities are determined in this context using the falling ball method in accordance with British Standard 188. Comparable values are obtained using manufacturer testing instructions analogous to British Standard 188, for example “CTM 0577” of the Dow Corning Corporation.

In a particular embodiment, (in particular, cyclic) siloxanes such as, for example, the products Dow Corning® 344, Dow Corning® 345, Dow Corning® 244, Dow Corning® 245, or Dow Corning® 246, having kinematic viscosities of up to 10,000 cSt determined at 25°, are used in accordance with the manufacturer's instructions.

Lastly, dialkyl carbonates, such as those described in detail in DE Application 197 101 54, to which reference is expressly made, are also oil components usable herein. Dioctyl carbonates, in particular di-2-ethylhexyl carbonate, are preferred oil components.

The agents contemplated herein can furthermore additionally contain alcohols that are miscible only to a limited extent with water.

“Miscible to a limited extent with water” is understood to mean alcohols that are soluble in water at about 20° C. at a proportion of no more than about 10 wt %, based on the mass of water.

In many cases, triols and in particular diols have proven particularly suitable. Alcohols having 4 to 20, in particular 4 to 10 carbon atoms are usable herein. Suitable alcohols can be saturated or unsaturated and linear, branched, or cyclic. Usable herein are, for example, butanol-1, cyclohexanol, pentanol-1, decanol, octanol, octenol, dodecenol, decenol, octadienol, dodecadienol, decadienol, ( )eyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprinyl alcohol, linoleyl alcohol, linolenyl alcohol, and behenyl alcohol, as well as Guerbet alcohols thereof, this listing being intended to be exemplary and not limiting in nature. The fatty alcohols derive, however, from preferably natural fatty acids; it is usually possible to proceed from an extraction from the esters of the fatty acids by reduction. Also usable are those fatty alcohol cuts that are generated by the reduction of naturally occurring triglycerides such as beef tallow, palm oil, peanut oil, colza oil, cottonseed oil, soybean oil, sunflower oil, and linseed oil, or from fatty acid esters resulting from transesterification products thereof with corresponding alcohols, and thus represent a mixture of different fatty alcohols.

2-Ethylhexandediol-1,3, butanol-1, cyclohexanol, pentanol-1, and 1,2-butanediol are preferred as alcohols. 2-Ethylhexanediol-1,3 in particular, but also butanol-1 and cyclohexanol, are particularly preferred.

In a further embodiment, emulsifiers can be used in the agents contemplated herein. Emulsifiers cause the formation, at the phase interface, of water-oil-stable adsorption layers that prevent the dispersed droplets from coalescing, and thereby stabilize the emulsion. Emulsifiers are therefore, like surfactants, constructed from a hydrophobic and a hydrophilic molecule part. Hydrophilic emulsifiers preferentially form o/w emulsions, and hydrophobic emulsifiers preferentially form w/o emulsions. An “emulsion” is to be understood as a droplet-like distribution (dispersion) of one liquid in another liquid, with the expenditure of energy to create stabilizing phase interfaces by means of surfactants. Selection of these emulsifying surfactants or emulsifiers is based on the substances to be dispersed and the respective external phase, and on the fineness of the emulsion particles. Additional definitions and properties of emulsifiers are found in H.-D. Dörfler, “Grenzflächen- and Kolloidchemie” [Interfacial and colloid chemistry], VCH Verlagsgesellschaft mbH, Weinheim, 1994. Emulsifiers usable herein are, for example:
addition products of 4 to 100 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂ to C₂₂ fatty acid mono- and diesters of addition products of 1 to 30 mol ethylene oxide with polyols having 3 to 6 carbon atoms, in particular with glycerol;
addition products of ethylene oxide and polyglycerol with methyl glucoside fatty acid esters, fatty acid alkanolamides, and fatty acid glucamides;
C₈ to C₂₂ alkyl mono- and oligoglycosides and ethoxylated analogs thereof, degrees of oligomerization from 1.1 to 5, in particular 1.2 to 2.0, and glucose as the sugar component, being preferred;
mixtures of alkyl(oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood as a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols called “mycosterols” are also isolated from fungi and yeasts.
Phospholipids. These are understood as principally the glucose phospholipids, which are obtained e.g. as lecithins, particularly phosphatidylcholines from, for example, egg yolk or plant seeds (e.g. soybeans).
Fatty acid esters of sugars and sugar alcohols, such as sorbitol;
polyglycerols and polyglycerol derivatives such as, for example, polyglycerol poly-12-hydroxystearate (commercial product Dehymuls® PGPH).
linear and branched fatty acids having 8 to 30 carbon atoms, and the Na, K, Ca, Mg, and Zn salts thereof.

The agents contemplated herein contain the emulsifiers preferably in quantities from about 0.1 to about 25 wt %, in particular about 0.1 to about 3 wt %, based on the respective total composition.

The agents can preferably contain at least one nonionogenic emulsifier having an HLB value from 8 to 18, according to the definitions set forth in the Römpp-Lexikon Chemie [Rompp chemical dictionary] (J. Falbe, M. Regitz, eds.), 10th edition, Georg Thieme Verlag Stuttgart, New York (1997), page 1764. Nonionogenic emulsifiers having an HLB value from 10 to 15 can be particularly preferred.

The agent is present as a water-containing agent. A “water-containing agent” as used herein contains at least 40 wt % water based on the weight of the total agent. The water-containing agent can be present in a variety of forms, for example as a lotion, oil-in-water emulsion, or water-in-oil emulsion.

The present disclosure also encompasses, however, those embodiments of the agent in which a multi-phase agent is present, which agent can be manufactured from two or more separately packaged starting preparations only directly before utilization. This embodiment can be preferred in the case of incompatible constituents. It has been found, surprisingly, that waving agents formulated in this manner result in a considerably increased waving effect for the same quantity of the respective keratin-reducing components. It is thus possible in this manner to achieve the waving performance attained with an agent formulated as set forth herein with a considerable decrease in the proportion of keratin-reducing substance, and thus with even less stress on the hair and scalp.

Two- and multi-phase systems usable herein are systems in which at least two separate, continuous phases are present. Examples of such systems are preparations that comprise the following phases:
an aqueous phase and a nonaqueous phase that are present separately from one another;
an aqueous phase and two nonaqueous mutually immiscible phases that are each present separately;
an oil-in-water emulsion and a nonaqueous phase present separately therefrom;
a water-in-oil emulsion and an aqueous phase present separately therefrom.

Systems in which only one continuous phase is present, for example pure oil-in-water or water-in-oil emulsions, are not two-phase systems for purposes herein.

It has furthermore proven advantageous if the agents contain a care-providing active substance, selected from protein hydrolysates and derivatives thereof.

Suitable protein hydrolysates are, in particular, hydrolysates of elastin, collagen, keratin, milk protein, and protein hydrolysates of silk, soy, almonds, peas, potatoes, oats, corn, and wheat. Plant-based products can be preferred in this context.

Suitable derivatives are, in particular, quaternized protein hydrolysates. Examples of this compound class are the products on the market under the designations Lamequat® L (CTFA name: Lauryldimonium Hydroxypropylamino Hydrolyzed Animal Protein; Grünau), Croquat® WKP, and Gluadin® WQ. The last-named product, which is plant-based, can be preferred.

The protein derivatives are contained in the agents preferably in quantities from about 0.1 to about 10 wt %, based on the total quantity of the agent. Quantities from about 0.1 to about 5 wt % are preferred.

The agents preferably furthermore contain at least one conditioning active substance.

Cationic polymers are preferably appropriate as conditioning active substances. These are, as a rule, polymers that contain a quaternary nitrogen atom, for example in the form of an ammonium group.

Preferred cationic polymers are, for example
quaternized cellulose derivatives such as those obtainable commercially under the names Celquat® and Polymer JR®. The compounds Celquat® H 100, Celquat® L 200, and Polymer JR® 400 are preferred quaternized cellulose derivatives.
Polysiloxanes having quaternary groups,
Polymeric dimethyldiallylammonium salts and copolymers thereof with esters and amides of acrylic acid and methacrylic acid. The products obtainable commercially under the names Merquat® 100 (poly(dimethyldiallylammonium chloride)) and Merquat® 550 (dimethyldiallylammonium chloride-acrylamide copolymer) are examples of such cationic polymers.
Copolymers of vinylpyrrolidone with quaternized derivatives of dialkylaminoacrylate and -methacrylate, such as e.g. vinylpyrrolidone-dimethylaminomethacrylate copolymers quaternized with diethyl sulfate. Such compounds are obtainable commercially under the names Gafquat® 734 and Gafquat® 755.
Vinylpyrrolidone-vinylimidazolinium methochloride copolymers, such as those offered under the Luviquat® name,
quaternized polyvinyl alcohol,
   as well as the polymers, known by the names
Polyquaternium 2,
Polyquaternium 17,
Polyquaternium 18, and
Polyquaternium 27,
   having quaternary nitrogen atoms in the main polymer chain.

Amphopolymers are also suitable as conditioning active substances. The general term “amphopolymers” covers amphoteric polymers, i.e. polymers that contain both free —COOH or SO₃H groups in the molecule and are capable of forming internal salts, zwitterionic polymers that contain quaternary ammonium groups and —COO⁻ or —SO₃⁻ groups in the molecule, and those polymers that contain —COOH or SO₃H groups and quaternary ammonium groups. One example of an amphopolymer usable herein is the acrylic resin obtainable under the name Amphomer®, which represents a copolymer of tert-butylaminoethyl methacrylate, N-(1,1,3,3-tetramethylbutyl)acrylamide, and two or more monomers from the group of acrylic acid, methacrylic acid, and simple alkyl esters thereof. Amphopolymers that are likewise preferred are made up of unsaturated carboxylic acids (e.g. acrylic and methacrylic acid), cationically derivatized unsaturated carboxylic acids (e.g. acrylamidopropyltrimethylammonium chloride), and optionally further ionic or nonionogenic monomers, as may be gathered for example from German Application 39 29 973 and the existing art cited therein. Terpolymers of acrylic acid, methyl acrylate, and methacrylamidopropyltrimonium chloride, such as those obtainable commercially under the name Merquat® 2001 N, as well as the commercial product Merquat® 280, are amphopolymers particularly preferred.

The cationic or amphoteric polymers are contained in the preparations preferably in quantities from about 0.1 to about 5 wt %, based on the total preparation.

Silicone gums, such as e.g. the commercial product Fancorsil® LIM-1, as well as anionic silicones such as e.g. the product Dow Corning® 1784, are further suitable as conditioning active substances.

Examples of cationic surfactants usable as conditioning active substances in the agents are, in particular, quaternary ammonium compounds. Ammonium halides are preferred, in particular chlorides and bromides, such as alkyltrimethylammonium chlorides, dialkyldimethylammonium chlorides, and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. The highly biodegradable quaternary ester compounds called “esterquats,” such as e.g. the methylhydroxyalkyldialkoyloxyalkylammonium methosulfates marketed under the trade names Dehyquart® and Stepantex®, can also be used.

Alkylamidoamines, in particular fatty acid amidoamines such as the stearylamidopropyldimethylamine obtained under the name Tego Amid® S 18, are notable not only for a good conditioning effect but also especially for their good biodegradability.

It can also be preferred to color the individual phases with dyes in order to achieve a particularly good visual appearance for the agent. These dyes are preferably soluble only in the aqueous phase, or only in at least one nonaqueous phase, in a quantity that allows a corresponding tint to be visible to the viewer. It is also possible to tint both the nonaqueous phase and the aqueous phase with different dyes, preferably of different colors. Coloring only of a nonaqueous phase is, however, preferred.

Further usual constituents of the agents are:
anionic surfactants such as, for example, soaps, alkyl sulfates, and alkyl polyglycol ether sulfates, salts of ethercarboxylic acids of the formula R—O—(CH₂—CH₂—O)ₓ—CH₂—COOH, in which R is a linear alkyl group having 10 to 22 carbon atoms and x=0 or is 1 to 16, acylsarcosides, acyltaurides, acylisethionates, succinic acid mono- and dialkyl esters, linear alkanesulfonates, linear alpha-olefinsulfonates, alpha-sulfo fatty acid methyl esters, and esters of tartaric acid and citric acid, alkylglycosides, or alcohols that represent addition products of approximately 2 to 15 molecules of ethylene oxide and/or propylene oxide with fatty alcohols having 8 to 22 carbon atoms.
Zwitterionic surfactants such as, for example, betaines and 2-alkyl-3-carboxymethyl-3-hydroxyethylimidazolines.
Ampholytic surfactants such as, for example, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, N-alkylaminopropionic acids, and alkylaminoacetic acids.
Nonionic surfactants such as, for example, addition products of 2 to 30 mol ethylene oxide and/or 0 to 5 mol propylene oxide with linear fatty alcohols having 15 to 22 carbon atoms, with fatty acids having 12 to 22 carbon atoms, and with alkylphenols having 8 to 15 carbon atoms in the alkyl group, C₁₂ to C₂₂ fatty acid mono- and diesters of addition products of 1 to 30 mol ethylene oxide with glycerol, C₈ to C₂₂ alkylmono- and oligoglycosides and ethoxylated analogs thereof, as well as addition products of 5 to 60 mol ethylene oxide with castor oil and hardened castor oil.
Nonionic polymers such as, for example, vinylpyrrolidone/vinyl acrylate copolymers, polyvinylpyrrolidone, and vinylpyrrolidone/vinylacetate copolymers.
Anionic polymers, such as polyacrylic and polymethacrylic acids, salts thereof, copolymers thereof with esters and amides of acrylic acid and of methacrylic acid, and derivatives thereof that are obtained by crosslinking with polyfunctional agents, polycarboxylic acids such as polyketo- and polyaldehydocarboxylic acids and salts thereof, as well as polymers and copolymers of crotonic acid with esters and amides of acrylic acid and of methacrylic acid, such as vinyl acetate-crotonic acid and vinyl acetate-vinyl propionate-crotonic acid copolymers.
Organic thickening agents such as agar-agar, guar gum, alginates, cellulose ethers such as methyl and methylhydroxypropyl cellulose, gelatins, pectins, and/or xanthan gum. Ethoxylated fatty alcohols, in particular those having a restricted homolog distribution, such as those marketed e.g. as a commercial product under the name Arlypon® F (Henkel), alkoxylated methylglucoside esters such as the commercial product Glucamate® DOE 120 (Amerchol), and ethoxylated propylene glycol esters such as the commercial product Antil® 141 (Goldschmidt), can be preferred organic thickening agents,
structuring agents such as glucose and maleic acid,
hair-conditioning compounds such as phospholipids, for example soy lecithin, egg lecithin, and kephalins,
perfume oils,
solubilizers such as ethanol, isopropanol, ethylene glycol, propylene glycol, glycerol, diethylene glycol, and ethoxylated triglycerides, as well as fatty alcohol ethoxylates and derivatives thereof,
anti-dandruff active substances such as climbazol, piroctone olamine, and zinc omadine,
active substances such as bisabolol, allantoin, panthenol, niacinimide, tocopherol, and plant extracts,
light protection agents,
consistency agents such as sugar esters, polyol esters, or polyol alkyl ethers,
fatty acid alkanolamides,
complexing agents such as EDTA, NTA, β-alaninediacetic acid, and phosphonic acids,
swelling and penetration substances such as PCA, glycerol, propylene glycol monoethyl ether, carbonates, hydrogen carbonates, guanidines, ureas, and primary, secondary, and tertiary phosphates,
opacifiers such as latex or styrene/acrylamide copolymers,
luster agents such as ethylene glycol mono- and distearate or PEG-3 distearate,
substantive dyes, and
propellants such as propane-butane mixtures, N₂O, dimethyl ether, CO₂, and air.

In another embodiment, a kit of parts encompasses
(i) at least one container containing an agent as described above, and
(ii) at least one container containing at least one fixing agent having a pH of at most about pH 7, encompassing at least one oxidizing agent.

Oxidizing agents are a constituent of the fixing agent contained in the kit of parts. “Oxidizing agents” for purposes herein are capable of oxidatively forming a disulfide bond from two thiol groups. Sodium bromate or potassium bromate, or hydrogen peroxide, are preferably suitable. Hydrogen peroxide is a particularly preferred oxidizing agent.

The fixing agent preferably contains at least about 50 wt % water.

The pH of H₂O₂-containing fixing agents is preferably about 2 to about 6, in particular about 2 to about 4. The H₂O₂ preparations preferably contain about 0.5 to about 15 wt %, in particular about 0.5 to about 3 wt % H₂O₂. Bromate-based fixing agents contain the bromates preferably in concentrations from about 1 to about 10 wt %, and the pH of these solutions is again adjusted preferably to about 4 to about 7.

A further embodiment is a method for permanent deformation of keratin-containing fibers, in particular human hair, in which
(i) the fibers are deformed with the aid of deformation aids after, before, or during step (ii),
(ii) an agent as described above is applied onto the fibers,
(iii) after a contact time Z1 the fibers are rinsed and optionally dried,
(iv)a fixing agent containing at least one oxidizing agent is then applied onto the fibers and is rinsed out again after a contact time Z2.

“Deformation aids” for purposes of the method herein can be
for example, hair curlers or rollers in the case of a permanent wave, or
aids for mechanical straightening, such as a comb or brush, a straightening board, or a heatable straightening iron in the case of hair straightening.

If the deformation aids, in particular curlers, are secured to the fibers for a longer period of time in the context of a permanent wave process, it is then useful to remove the deformation aids before step (iii) or after step (iv). It can be advantageous in this context to leave the deformation aids in the hair during step (iv), then remove them, and then repeat step (iv) as a so-called post-fixing step (v).

In a preferred embodiment, the keratin-containing fibers are moistened before step (i). This can be done by spraying the fibers with a liquid, preferably with water. Preferably, the fibers are shampooed with a conventional shampoo before step (i), rinsed, and then rubbed with a hand towel. Palpable residual moisture remains in the hair after the toweling step is completed.

“Mechanical straightening” is understood herein as a stretching of the curly fibers along their longest physical extent.

Contact time Z1 is preferably about 5 to about 60 minutes, particularly preferably about 10 to about 30 minutes.

Contact time Z2 is preferably about 1 to about 30, particularly preferably about 5 to about 20 minutes.

The fixing agents usable in the method, and preferably usable fixing agents, are those described above.

The Examples that follow are intended to explain the various embodiments further.

### EXAMPLES

The formulations of Examples 1 to 6 below were produced by mixing the raw materials. Table 1 encompasses two preparations as contemplated herein labeled E1 and E2, and one comparison composition labeled with a V. Each example formulation was tested on ten test subjects using a half-side test, performing the following permanent wave process:

All of the hair on the head is moistened with water and rubbed with a hand towel.

The hair is parted with a comb into a left and a right half. The hair is then wound onto rollers. The left half is treated with 30 mL of preparation W, and the right half-side with 30 mL of the corresponding comparison formulation V of the respective Example.

After a contact time of 20 minutes, the hair is rinsed with water and 25 mL of a fixing agent in accordance with Table 2 below is applied onto the entire hair area.

After a contact time of 10 minutes the rollers are removed and the hair is thoroughly rinsed.

The waving result achieved, and the state of the scalp, are then visually evaluated, and data regarding the feel of the treated scalp are gathered by questioning the test subject.

The following raw materials were used in the example formulations:
¹ Aqueous solution with 71 wt % active substance
² Aqueous solution with 83 wt % active substance
³ Aqueous 25% solution
⁴ 1-Hydroxyethane-1,1-diphosphonic acid, aqueous solution with approx. 60% active substance (INCI name: Etidronic Acid) (Solutia)
⁵ Trimethylhexadecylammonium chloride (approx. 24 to 26% active substance; INCI name: Aqua (Water), Cetrimonium Chloride) (Solutia)
⁶ N,N-Dimethyl-N-cocalkylamine-N-oxide (approx. 30% active substance; INCI name: Cocamine Oxide) (Akzo Nobel)

TABLE 1
Waving agents

| Ingredients | E1 | | E2 | | V1 | |
|---|---|---|---|---|---|---|
| Ammonium thioglycolate ¹ | — | | — | | 18.0 | g |
| 2-Ammonioethanol thioglycolate ² | 21.4 | g | 22.0 | g | — | |
| Sodium bicarbonate | 3.0 | g | 5.0 | g | — | |
| Ammonium bicarbonate | — | | — | | 8.8 | g |
| Ammonia, 25% ³ | — | | — | | 1.8 | g |
| Turpinal SL ⁴ | 0.1 | g | 0.1 | g | 0.3 | g |
| Water | to 100 | g | to 100 | g | to 100 | g |

TABLE 2
Fixing agents

| Raw material | Quantity (wt %) |
|---|---|
| Phosphoric acid (85 wt % in water) | 0.95 |
| Hydrogen peroxide | 2.00 |
| Methylparaben | 0.04 |
| Dehyquart A CA ⁵ | 0.30 |
| Polyquaternium-6 | 0.50 |
| Aromox MCD W ⁶ | 3.00 |
| Perfume | 0.30 |
| Distilled water | to 100 |

All three formulations produced an acceptable waving result. When composition V1 was used, however, individual reddened areas were observed on the treated scalp region. Formulations E1 and E2 yielded improved compatibility, with reduced reddening and irritation of the skin as well as reduced odor impact.

1. A water-containing agent for the permanent deformation of keratin-containing fibers, the water-containing agent comprising:
a) about 0.5 to about 20 wt % cysteine; and
b) about 0.5 to about 10 wt % of a (C₂ to C₆) alkanolamine as an alkalizing agent,
wherein the water-containing agent has a pH from about 8.0 to about 8.5 and contains no more than about 0.1 mmol ammonia per 100 g of the water-containing agent.

2. The water-containing agent according to claim 1, wherein the keratin-containing fibers are human hair.

3. The water-containing agent according to claim 1, containing about 1.0 to about 15.0 wt % cysteine.

4. The water-containing agent according to claim 3, containing about 1.5 to about 10 wt % cysteine.

5. The water-containing agent according to claim 4, containing about 2.0 to about 7.5 wt % cysteine.

6. The water-containing agent according to claim 5, containing about 2.25 to about 5 wt % cysteine.

7. The water-containing agent according to claim 1, wherein the water-containing agent comprises cysteine in a quantity from about 40 mmol to about 130 mmol, based on 100 g of the water-containing agent.

8. The water-containing agent according to claim 1, wherein the water-containing agent comprises cysteine in a quantity from about 50 mmol to about 120 mmol, based on 100 g of the water-containing agent.

9. The water-containing agent according to claim 1, wherein the (C₂ to C₆) alkanolamines of the alkalizing agent are chosen from 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropane-1,3-diol (in particular from among 2-aminoethan-1-ol, 2-amino-2-methylpropan-1-ol, and 2-amino-2-methyl-propane-1,3-diol).

10. The water-containing agent according to claim 1, further comprising a compound that having a HCO₃⁻ ion and/or a CO₃²⁻ ion.

11. The water-containing agent according to claim 1, further comprising no more than about 0.1 mmol (NH₄⁺) ions.

12. The water-containing agent according to claim 1, wherein the pH is in a range of from about 8.1 to about 8.5.

13. The water-containing agent according to claim 12, wherein the pH is in the range of from about 8.1 to 8.4.

14. The water-containing agent according to claim 13, wherein the pH is in the range of from about 8.1 to about 8.3.

15. A kit of parts comprising:
(i) a first container;
(ii) a water-containing agent within the first container, the water-containing agent for the permanent deformation of keratin-containing fibers, wherein the water-containing agent comprises:
a. about 0.5 to about 20 wt % cysteine; and
b. about 0.5 to about 10 wt % of a (C₂ to C₆) alkanolamine as an alkalizing agent,
wherein the water-containing agent has a pH from about 8.0 to about 8.5 and contains no more than about 0.1 mmol ammonia per 100 g of the water-containing agent; and
(iii) a second container containing a fixing agent having a pH of no greater than about pH 7 comprising an oxidizing agent.

16. A method for permanent deformation of keratin-containing fibers, the method comprising the steps of:
applying a water-containing agent to the keratin-containing fibers, the water-containing agent comprising:
a. about 0.5 to about 20 wt % cysteine; and
b. about 0.5 to about 10 wt % of a (C₂ to C₆) alkanolamine as an alkalizing agent,
wherein the water-containing agent has a pH from about 8.0 to about 8.5 and contains no more than about 0.1 mmol ammonia per 100 g of the water-containing agent;
deforming the keratin-containing fibers with the aid of deformation aids after, before, or during the applying the water-containing agent;
after a contact time Z1, rinsing and optionally drying the keratin-containing fibers;
applying onto the keratin-containing fibers a fixing agent having a pH of at most about 7, wherein the fixing agent comprises an oxidizing agent; and
rinsing the fixing agent from the keratin-containing fibers after a contact time Z2.

17. The method according to claim 16, wherein applying the water-containing agent comprises applying the water-containing agent to human hair.
